(19) United States
(12) Patent Application Publication
(10) Pub. No.: US 2024/0307262 A1
(43) Pub. Date: Sep. 19, 2024

(54) ROTATIONAL ACCESSORY FOR A PERCUSSION MASSAGER

(71) Applicant: PlayMakar Inc., Fort Worth, TX (US)

(72) Inventors: Michael James Williams, Colleyville, TX (US); Federico G. Vierheller, Fort Worth, TX (US); Wesley A. Coleman, N. Richland Hills, TX (US)

(73) Assignee: PlayMakar Inc., Fort Worth, TX (US)

(21) Appl. No.: 18/183,463

(22) Filed: Mar. 14, 2023

(51) Int. Cl. A61H 23/00; A61H 15/00; A61N 5/06

(52) U.S. Cl. CPC A61H 23/006; A61H 15/0085; A61N 5/0613; A61H 2015/0042; A61H 2201/0107; A61H 2201/0207; A61H 2201/0228; A61H 2201/10; A61H 2201/1671; A61H 2201/1685; A61H 2201/1692; A61H 2201/5048; A61H 2201/5084; A61N 2005/0652; A61N 2005/0659; A61N 2005/0663

(57) ABSTRACT

A tool for a percussive massaging device having a percussive massager, including a tool receiver, includes an attachment which is removably attached within the tool receiver to facilitate rotation and repositioning of the tool within the tool receiver and a tool head rotatably connected the attachment post, wherein an orientation a longitudinal axis of the tool head is repositioned by removing, rotating and repositioning the attachment post within the tool receiver. The tool head having a body-contacting surface with a particular indentation pattern and components for facilitating heating to provide a thermal effect to the user's body.

### CROSS-REFERENCE TO RELATED APPLICATIONS

The following applications and patent are incorporated herein by reference in their entireties: U.S. patent application Ser. No. 17/223,840 entitled PERCUSSIVE MASSAGER ROTATIONAL ACCESSORY, filed Apr. 6, 2021; U.S. patent application Ser. No. 17/229,860 entitled VARIABLE STROKE PERCUSSIVE MASSAGE DEVICE, filed Apr. 13, 2021, now U.S. Pat. No. 11,253,423; U.S. patent application Ser. No. 17/508,954 entitled CONSTRAINED AND REPOSITIONABLE PERCUSSIVE MASSAGE DEVICE TOOL AND TOOL RECEIVER, filed Oct. 22, 2021; U.S. patent application Ser. No. 17/524,732 entitled PERCUSSION MASSAGER HAVING VARIABLE AND SELECTABLE STROKE LENGTH filed Nov. 11, 2021; and U.S. patent application Ser. No. 17/714,140 entitled SELECTABLE, CONFIGURABLE AND INTERCHANGEABLE MASSAGE TOOL HEAD SYSTEM FOR PERCUSSION MASSAGE DEVICES filed Apr. 5, 2022, and U.S. patent application Ser. No. 17/740,143 entitled SYSTEM AND METHOD FOR LOCKING PERCUSSION SETTINGS ON A MASSAGE DEVICE filed May 9, 2022, each of which is commonly owned and list overlapping inventors.

### FIELD OF THE INVENTION

The embodiments related generally to percussive massaging devices, and more particularly to a novel tools and tool configurations for a percussive massaging device.

### BACKGROUND

Vibratory or percussive massaging devices typically include a tool head that is adapted for contacting a person's body and delivering a therapeutic effect thereto. But heretofore the movement of the tool head has been confined to one dimension, that is, forward and backward along a longitudinal axis of the tool head. Further, many massaging devices of the prior art include a single tool head that is not easily directed to deliver a therapeutic effect to the person's body along multiple dimensions or directions. Other effects, such as heating or cooling of the person's body are also limited in the prior art. Therefore, there is a need for a tool head for a percussive massaging device that provides a therapeutic effect to the person's body along multiple dimensions and directions. Such a needed invention would be easily interchanged with a tool head that provides a different therapeutic effect. Further, such a needed device would be relatively simple to manufacture and intuitive to use. The present invention accomplishes these objectives.

### SUMMARY

In a first non-limiting embodiment, a tool for a percussive massaging device having a percussion massager, including a tool receiver, includes: a removable attachment post having a proximal end and a distal end and a longitudinal axis, the attachment post being adapted for selective attachment at its proximal end to the tool receiver of the percussive massaging device; and a dual-prong fork having a rotatable tool head inserted therebetween at a first end thereof, the dual-prong fork being connected at a second end thereof to the distal end of the attachment post, wherein an orientation of a longitudinal axis of the tool head is repositionable.

In a second non-limiting embodiment, a tool head for use with a percussive massaging device, includes: a rotatable wheel having at least one body-contacting surface having a predetermined pattern of indentations thereon; means for determining when the rotatable wheel is moving; means for generating heat through at least a portion of the at least one body-contacting surface; and means for generating light to be emitted from within the rotatable wheel.

### BRIEF DESCRIPTION OF THE DRAWINGS

FIGS. 1a and 1b show a first exemplary percussive massage device having a tool head in a first orientation (FIG. 1a) and a second orientation (FIG. 1b) in accordance with one embodiment herein;

FIGS. 2a and 2b is a detailed, exploded view of a tool head in accordance with one embodiment herein;

FIG. 3 shows a second exemplary percussive massage device having a second tool head in accordance with one embodiment herein; and

FIGS. 4a, 4b, 4c, 4d, and 4e show tool heads with different textured pattern surfaces or non-patterned surfaces.

### DETAILED DESCRIPTION

Illustrative embodiments are described below. The following explanation provides specific details for a thorough understanding of and enabling description for these embodiments. One skilled in the art will understand that the embodiments may be practiced without such details. In other instances, well-known structures and functions have not been shown or described in detail to avoid unnecessarily obscuring the description of the embodiments.

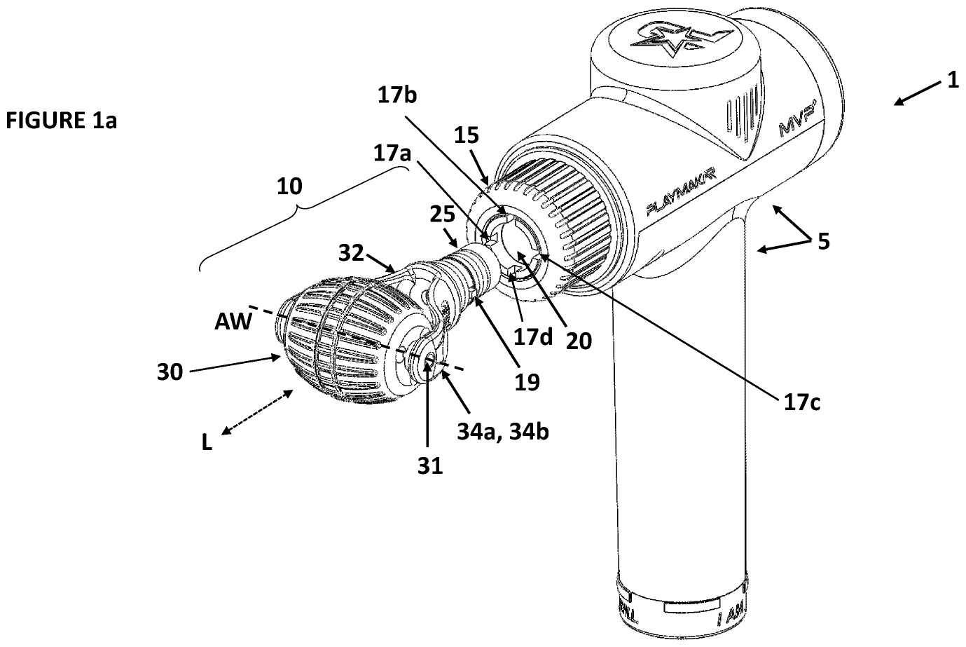

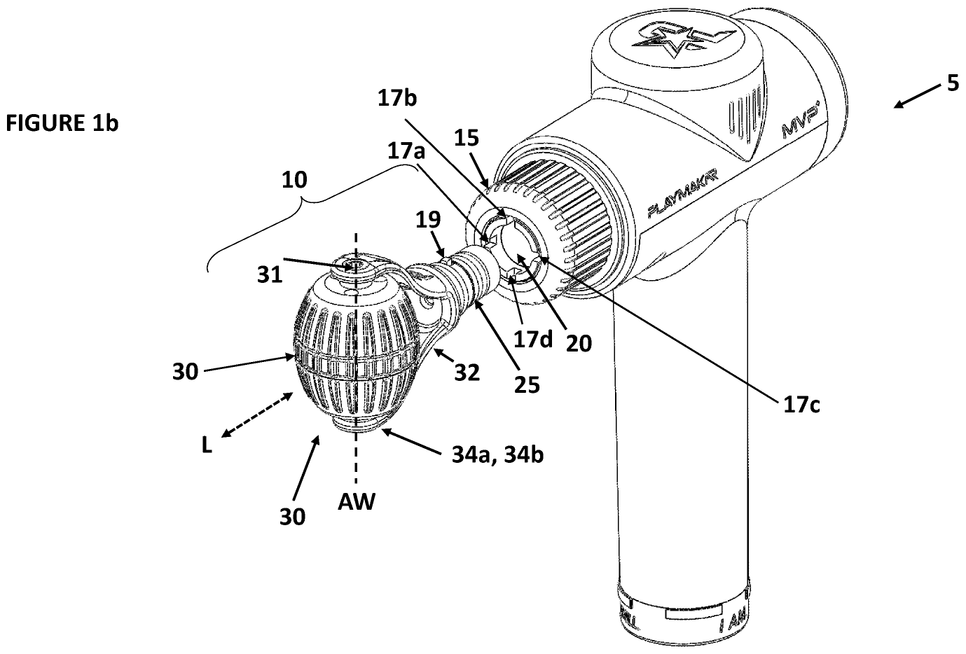

In FIG. 1a, a first exemplary percussive massage device 1 in accordance with a preferred embodiment includes a percussion massager 5 and tool 10 arranged in a first position when two keys 19 (only one shown) engage with two keyways (17a, 17c) located opposite to one another within the entrance of the tool receiver 15. In FIG. 1b, the tool 10 is arranged in a second position when the two keys 19 (only one shown) engage with the other two keyways (17b, 17d) located opposite to one another within the entrance of the tool receiver 15. Accordingly, the attachment post 25 can be clocked at or rotated at 90 and 180 degrees with the piston to align the tool head 30 to reach various muscles.

The first exemplary tool 10, further includes an interchangeable tool head 30 which is rotatably fixed at a far end (e.g., distal end) of an attachment post 25 and has at least one body-contacting surface. More particularly, the tool head 30 is attached at either end thereof to a fork 32 fixed at a first end thereof to the attachment post 25 and terminating at two opposing fork ends 34a, 34b at a second end thereof. In the present embodiment, the interchangeable tool head 30 is a wheel which is rotationally mounted at opposing sides thereof to the opposing distal fork ends 34a, 34b along axis 31. As such, the interchangeable tool head 30, e.g., wheel, is free to rotate AW about the wheel axis 31 along a person's body as the interchangeable tool head 30 additionally produces the percussive therapeutic effect along a longitudinal axis L of the attachment post 25 driven by the piston 20.

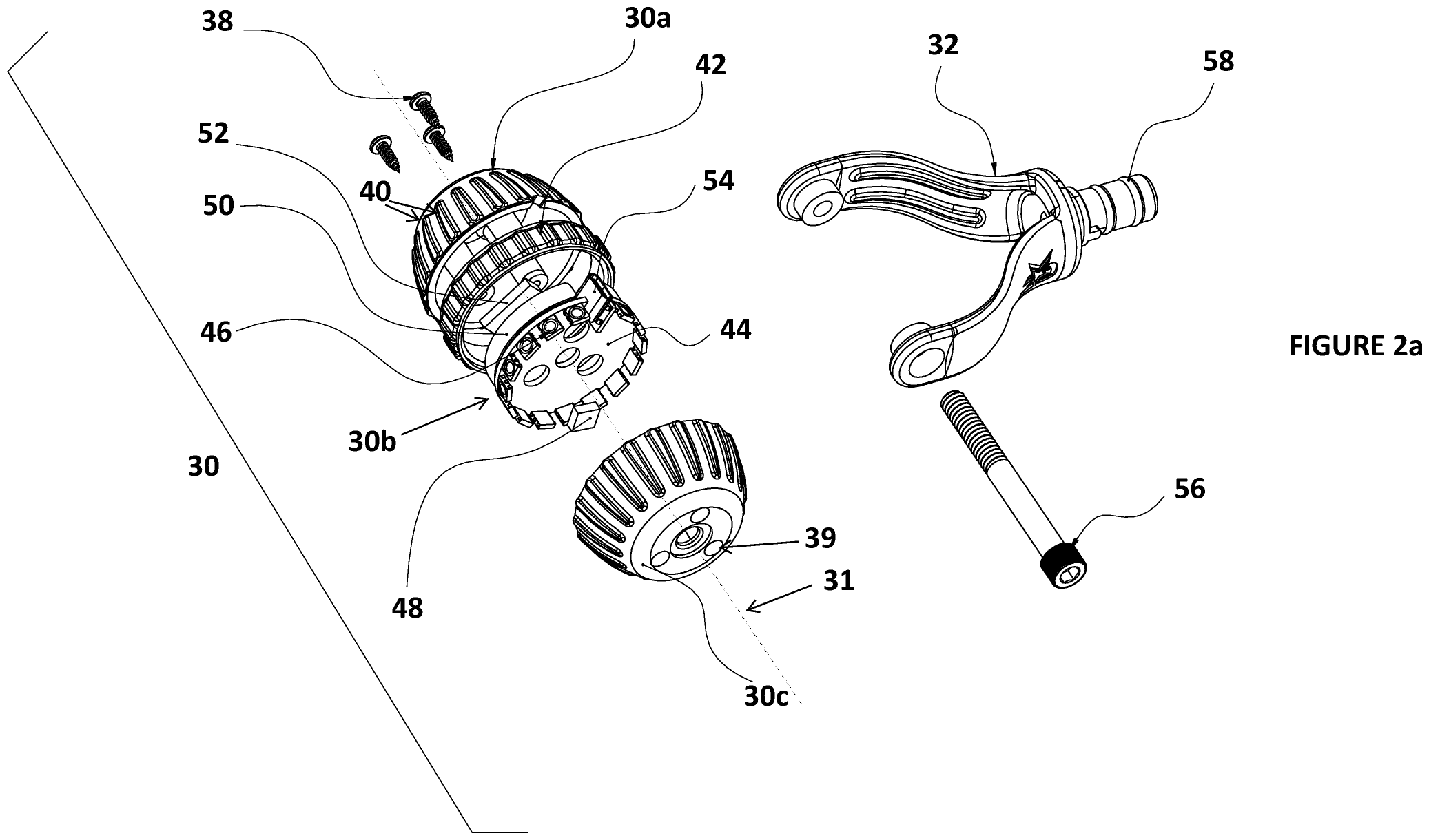

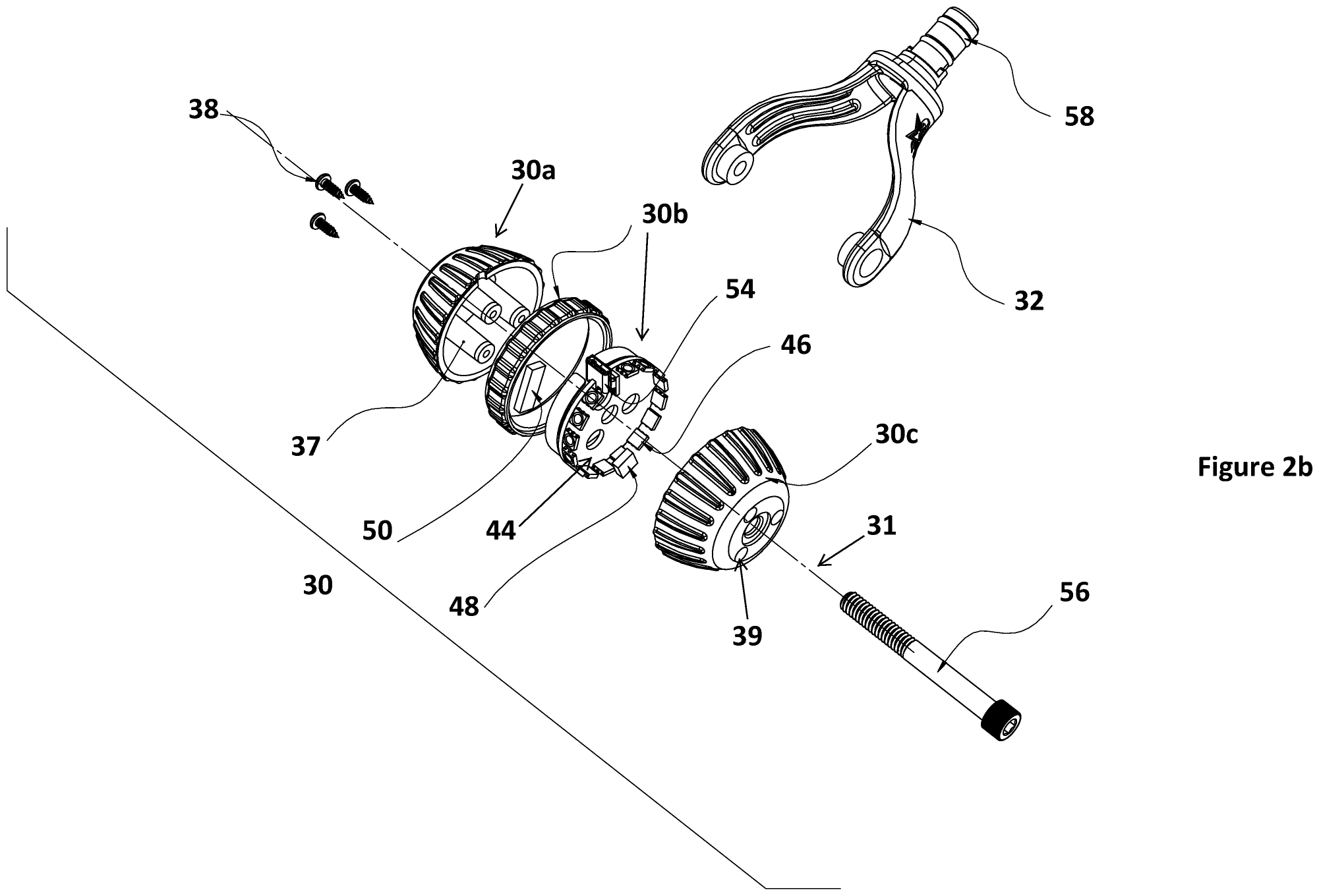

In FIGS. 2a and 2b, detailed, exploded views of an exemplary tool 10 are shown. The wheel of exemplary tool head 30, is shown in three parts, 30a, 30b, 30c. The parts are connected together during use by self-tapping screws (x3) 38 through. External tool head parts 30a, 30b and 30c include a surface ribbing pattern 40 which contacts the user's body during use. Tool head part 36b includes an interior component formed of: printed circuit (PC) board 44, LEDs 46, an accelerometer 48, a heater 50, a battery 52 and a USB port connector 54. Additionally, tool head part 30b includes an outer portion formed of: a transparent window section 42 which runs the circumference of the tool head 30 and is formed of a clear material such as Polyethylene terephthalate (PET) plastic; and a full or partial slot (not shown) for receiving USB port connector 54 therethrough when the tool head 30 is assembled so that connector 54 is accessible. The parts of the assembled tool head 30 are attached by self-tapping screws 38 and screw boss with countersink holes (x3) 37 into mating bosses (x3) 39. And the tool head 30 is secured rotatably attached to fork 32 by bolt 56. Tool 10 further includes o-ring(s) 58 for fitting the fork 32 to attachment post 25 (not shown). Tool head parts 30a and 30c may be composed of black ABS.

When assembled and in use. first exemplary percussive massage device 5 operates to provide heating to at least a portion of the assembled tool head 30 and to the user's body. Operating through PC board 44, heater 50 is activated when it receives a signal from PC board 44 that the accelerometer 48 detects motion (of the tool head 30). Alternatively, or in addition thereto, the PC board 44 also signals activation of the LED lights 46 when the accelerometer 48 detects motion (of the tool head 30). When assembled, the heater 50 of tool head part 30b aligns beneath the portion of tool head part 30a that is just beyond the transparent window section 42, while the LEDs 46 align beneath the transparent window section 42. When motion stops, the accelerometer 48 sends another signal to the PC board 44 to turn off the LED lights 46 and heater 50. Also when assembled, the USB port connector 54 fits through the slot in tool head part 30b and allows the battery 52 to be charged when the PC board 44 is not in use.

The signal indicating motion of the tool head 30 could be triggered by alternative means including a manual switch or voice activation.

In a preferred embodiment, the heater 50 is a flexible heater mainly composed of two elements, the film, and the heater. The heater and the film are bonded together using a process called heat sealing. Heat-sealing is a process that uses heat and pressure to join two materials together. During the process, the heat melts the surfaces of the material, allowing them to bond together.

The material used in flexible heater film to create heat is usually metal, such as copper, aluminum, or stainless steel. The metal is typically in the form of a wire or strip, which is then embedded in a polyimide film. When an electric current is passed through the metal, it generates heat and this heat is transferred to the polyimide film. The most commonly used heater materials are copper, aluminum, stainless steel, graphite, and ceramic. Copper is a good conductor of electricity and is often used in flexible heaters due to its low cost and excellent heat transfer properties. Aluminum is lightweight and easy to form into complex shapes. Stainless steel is strong and durable, making it a popular choice for flexible heaters. Graphite is an excellent heat conductor and is often used in lightweight heaters. Ceramics is a heat-resistant material that is often used for high-temperature applications.

The most commonly used film materials for flexible heaters are polyimide, Kapton, and PTFE. Polyimide is a heat-resistant plastic that can withstand temperatures up to 500° F. (260° C.). Kapton tape is a high-temperature, flame-resistant tape made from polyimide film. PTFE (polytetrafluoroethylene) is a heat-resistant plastic with excellent electrical insulation properties. The average thickness of a flexible heater is typically between 0.2 mm and 0.5 mm. However, one skilled in the art will appreciate that the exact thickness of the heater will depend on the application and the specific materials used.

The LEDs 46 may be selected from the group consisting of red (or other color) LED light, a near infrared LED or an infrared LED or the LEDs could be combinations of these. The LEDs 46 may work in combination with the heater 50 to provide a thermal effect to the body of the user of the percussive massage device 1. The LEDs are placed in an annular array emitting out of tool head part 30b as shown in FIG. 2.

Current pulsation of infrared LEDs is known to make a difference in muscle penetration and health benefits. Pulsed infrared light has been shown to penetrate deeper into the body than continuous infrared light, providing more effective muscle relaxation and pain relief.

Additionally, research has shown that pulsed infrared light can help increase circulation, reduce inflammation, and improve body elasticity and collagen production. Generally, a frequency of 10-20 Hz is most commonly used for muscle relaxation and pain relief. Higher frequencies (up to 50 Hz) can be used for increased circulation and inflammation reduction, while lower frequencies (down to 1 Hz) can be used for improved body elasticity and collagen production.

The type of LED used for infrared applications depends on the application and desired power output. Generally, gallium arsenide (GaAs) and gallium phosphide (GaP) LEDs are the most commonly used for infrared applications. These LEDs are usually available in the range of 850 nm to 950 nm wavelengths and can produce powers up to several watts. The type of LED used for near-infrared is generally available in the range of 750-1100 nm wavelengths and can produce powers up to several watts.

Alternatively, the LEDs 46 may be used to emit light of one or more colors for decorative effect.

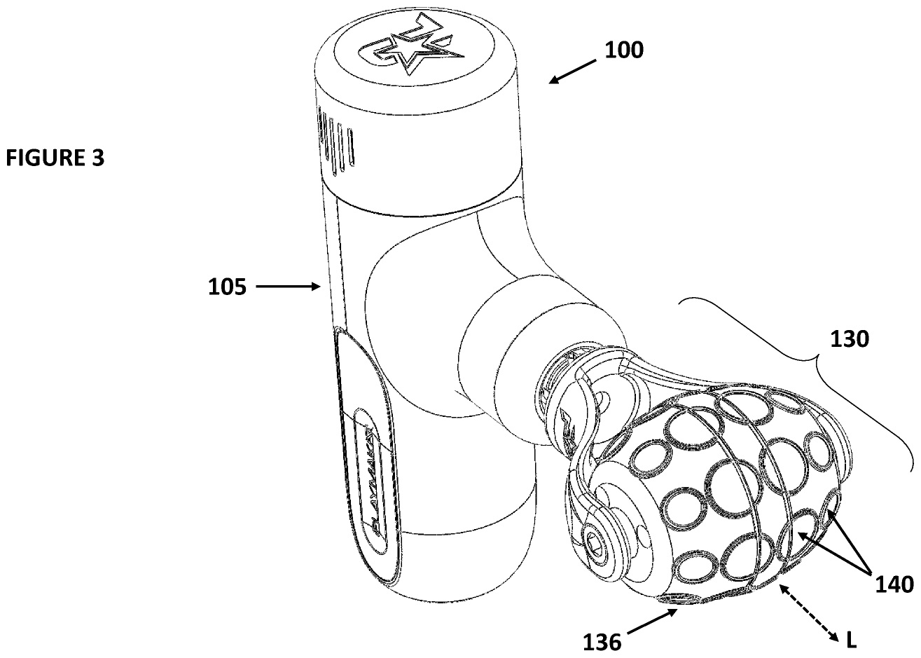

In FIG. 3, a second exemplary percussive massage device 100 in accordance with a preferred embodiment includes a percussion massager 105 and tool 110, wherein the percussion massager 105 is of a more compact or mini design as compared to percussion massager 5. Additionally, the tool head 130 includes a spherical pattern body-contacting surface 140 which provides a user with a different feel during use. All other features and components of the tool 110 and tool head 130 are the same as those described with respect to the first exemplary percussive massage device 1 as described above with reference to FIGS. 1a, 1b, 2a and 2b.

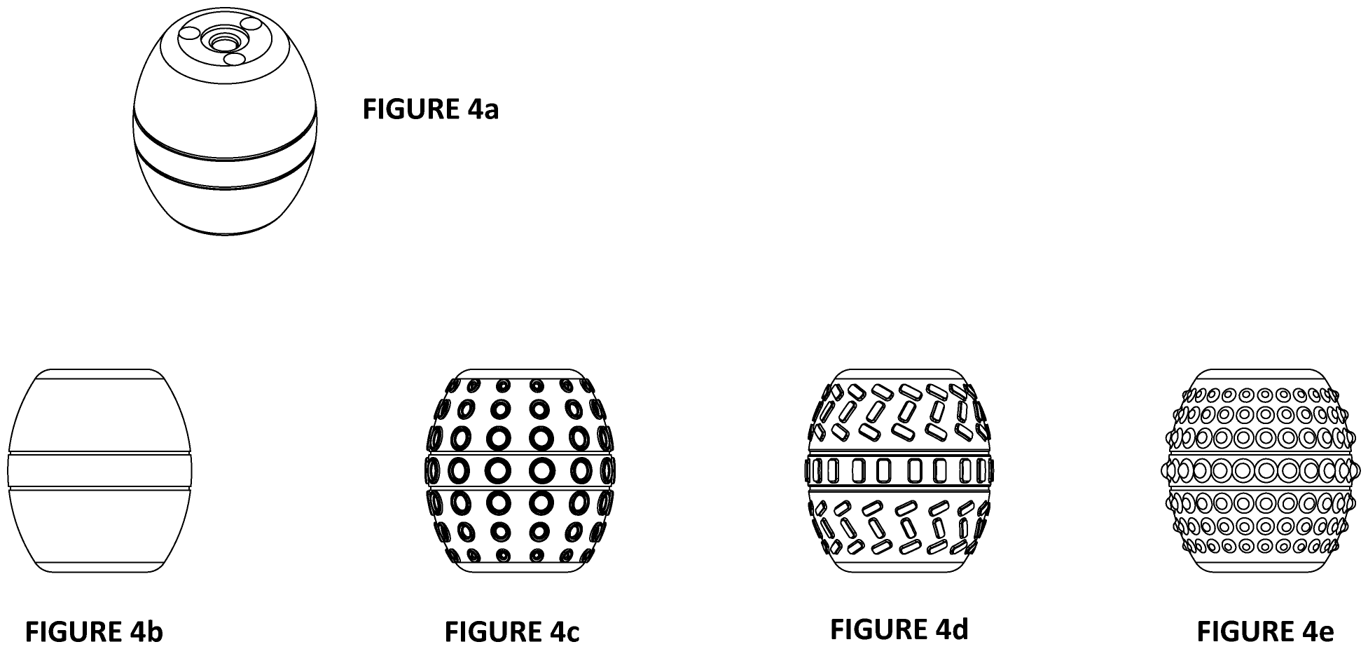

FIGS. 4a and 4b show a side and isometric view of an exemplary assembled tool head that does not include a textured pattern. FIGS. 4c to 4e show exemplary assembled tool heads having different textured patterns from that of tool head 30. The other components of the assembled tool heads shown in FIGS. 4a to 4e are identical to those described above with respect to tool head 30 and a user may interchange assembled heads with no texture or a different texture in the tool 10 as desired.

While a particular form of the embodiments has been illustrated and described, it will be apparent that various modifications can be made without departing from the spirit and scope of the embodiment. Accordingly, it is not intended that the embodiments be so limited.

1. A tool for a percussive massaging device having a percussion massager, including a tool receiver, the tool comprising:
a removable attachment post having a proximal end and a distal end and a longitudinal axis, the attachment post being adapted for selective attachment at its proximal end within the tool receiver of the percussive massaging device; and
a dual-prong fork having a rotatable tool head inserted therebetween at a first end thereof, the dual-prong fork being connected at a second end thereof to the distal end of the attachment post, wherein an orientation of a longitudinal axis of the tool head is repositionable by rotating the removable attachment post within the tool receiver.

2. The tool according to claim 1, wherein the attachment post includes at least one key for engaging with at least one keyway within the tool receiver and further wherein, the attachment post is removably attached within the tool receiver to facilitate rotation and repositioning of the tool within the tool receiver.

3. The tool according to claim 1, wherein the attachment post is capable of being removed, rotated at 90, 180 or 270 degrees, and re-attached within the tool receiver.

4. The tool according to claim 1, wherein the rotatable tool head has at least one body-contacting surface having a predetermined pattern of indentations thereon.

5. The tool according to claim 4, wherein the predetermined pattern of indentations is selected from the group consisting of ribs or ovals or textures.

6. The tool according to claim 1, wherein the rotatable tool head is interchangeable.

7. The tool according to claim 1, wherein the rotatable tool head is hollow.

8. The tool according to claim 4, further comprising:
means for determining when the rotatable tool head is moving;
means for generating heat through at least a portion of the at least one body-contacting surface; and
means for generating light to be emitted from within the rotatable tool head.

9. The tool according to claim 8, wherein the tool head includes a transparent, circumferential window in approximately the center of the tool head and further wherein internally generated light is emitted through the transparent, circumferential window.

10. The tool according to claim 8, wherein the means for determining when the rotatable tool head is moving is selected from the group consisting of an accelerometer, a switch or voice activation.

11. The tool according to claim 8, wherein the means for generating heat through at least a portion of the at least one body-contacting surface is a heating element.

12. The tool according to claim 8, wherein the means for generating heat through at least a portion of the at least one body-contacting surface is at least one of a red light LED, a near infrared LED or an infrared LED.

13. A tool head for use with a percussive massaging device, the tool head comprising:
a rotatable wheel having at least one body-contacting surface having a predetermined pattern of indentations thereon;
means for determining when the rotatable wheel is moving;
means for generating heat through at least a portion of the at least one body-contacting surface when it is determined that the rotatable wheel is moving; and
means for generating light to be emitted from within the rotatable wheel.

14. The tool head according to claim 13, further comprising:
a transparent, circumferential window in approximately the center of the wheel and further wherein internally generated light is emitted through the transparent, circumferential window.

15. The tool head according to claim 13, wherein the means for determining when the rotatable wheel is moving is selected from the group consisting of an accelerometer, a switch or voice activation.

16. The tool head according to claim 13, wherein the means for generating heat through at least a portion of the at least one body-contacting surface is a heating element.

17. The tool head according to claim 13, wherein the means for generating heat through at least a portion of the at least one body-contacting surface is at least one of a red light LED, a near infrared LED or an infrared LED.

18. The tool head according to claim 13, wherein the predetermined pattern of indentations is selected from the group consisting of ribs or ovals.

19. The tool head according to claim 13, further comprising a rechargeable battery.

20. The tool head according to claim 19, further comprising a USB port connector for use in recharging the rechargeable battery.
